(19) United States
(12) Patent Application Publication
(10) Pub. No.: US 2006/0068469 A1
(43) Pub. Date: Mar. 30, 2006

(54) Bacterial vector systems

(75) Inventors: Shelley M. Payne, Austin, TX (US); Jaquelin P. Dudley, Austin, TX (US); Stefan S. Seliger, Berkeley, CA (US); Zhengyu Feng, Austin, TX (US)

Correspondence Address: FULBRIGHT & JAWORSKI, LLP, 1301 MCKINNEY, SUITE 5100, HOUSTON, TX, 77010-3095, US

(73) Assignee: Research Development Foundation, Carson City, NV (US)

(21) Appl. No.: 11/205,883

(22) Filed: Aug. 17, 2005

Related U.S. Application Data
(60) Provisional application No. 60/602,276, filed on Aug. 17, 2004.

(51) Int. Cl. C12P 21/06; C12N 9/00; C12N 1/21; C07K 14/54; C07K 14/53; C07K 14/52; C07K 14/475

(52) U.S. Cl. 435/69.1; 435/252.3; 435/471; 530/350; 435/183; 530/351; 530/388.1

(57) ABSTRACT

The present invention provides bacterial vectors and fusion proteins containing a TTSS polypeptide, compositions of such fusion proteins including polynucleotides, and methods of delivering one or more genes into a target cell that involve contacting the cell with a composition that includes such a fusion protein. Compositions and methods of gene delivery that involve a bacterium and a TAT, Antp, or HSV VP22 polypeptide are also disclosed. The invention also concerns methods of delivering one or more genes into a target cell utilizing a bacterium capable of becoming internalized within the cell, wherein the bacterium includes one or more genes targeted for delivery to the cell, a gene encoding an RNA polymerase, and a gene that causes lysis of the bacterium.

This application claims priority to U.S. Provisional Patent Application Ser. No. 60/602,276 filed Aug. 17, 2004, which is incorporated herein by reference in its entirety.

Supported by grants from THECB, ARP 003658-0219-1997 and ARP 003658-0475-2001.

### BACKGROUND OF THE INVENTION

1. Field of the Invention

The present invention relates generally to the fields of protein chemistry, gene therapy, and microbiology. More particularly, it concerns compositions and methods that include bacterial vectors for delivering one or more polynucleotides to a target cell.

2. Description of Related Art

Bacterial type III secretion systems (TTSS) are a class of specialized protein secretion systems that deliver bacterial proteins directly into host cells (reviewed in He, 1998). Bacterial pathogens that utilize this system are known to be responsible for a number of diseases in plants, animals, and humans, such as rice leaf blight in plants and diarrhea in animals and humans. These pathogens are very diverse in their taxonomy, host range, and disease symptoms caused. A unique feature of the TTSS is the ability to deliver bacterial virulence proteins directly into host cells (Rosqvist et al., 1994). This protein delivery mechanism enables bacterial pathogens to gain access to a vast number of host targets, which would have been inaccessible if bacterial virulence proteins were delivered only to the surface of host cells.

Different bacteria use TTSS for different purposes. For example, plant pathogenic bacteria is believed to use a TTSS to secrete virulence proteins that cause leakage of plant nutrients to the extracellular space of infected tissues (Sigee and AL-Rabaee, 1986). Intracellular pathogens such as Salmonella spp. and Shigella spp. use TTSS for invasion of host cells (Galan and Bliska, 1996; Menard et al., 1996). In Yersinia spp. the TTSS is not used for invasion, but is used to resist uptake of bacteria by both phagocytic and non-phagocytic cells when bacteria are extracellular (Simonet et al., 1992).

Bacteria-mediated gene transfer is an alternative strategy to conventional gene therapy. Bacteria exhibit several unique properties such as tissue tropism and cell-to-cell spread. Thus, bacteria-mediated nucleic acid transfer has the potential for use in targeting tissue layers that are inaccessible to the conventional gene therapy vectors.

Typically, gene therapy is the treatment or prophylaxis of disease in a subject based on the transfer of genetic material (DNA/RNA), e.g., therapeutic polynucleotides. For example, gene therapy can be achieved either by direct administration of a virus encoding a therapeutic polynucleotide or indirectly through the introduction of genetically engineered cells to a subject.

A large number of disease states may be treated by the administration of various therapeutic polynucleotides, which may encode polypeptides including tumor suppressors, lymphokines, interferons, growth factors, tissue plasminogen activator, insulin, erythropoietin, thymidine kinase, and the like. Moreover, the selective delivery of therapeutic polynucleotides encoding toxic peptides to diseased, hyperplastic, or neoplastic cells can have major therapeutic benefits. The tremendous promise of conventional gene therapy is potentially limited due to a number of factors including inefficiency of gene transfer and limited DNA or RNA capacity of viruses or other vectors. Additionally, gene therapy vectors can be difficult to prepare and purify in large quantities.

Furthermore, the clinical application of conventional gene therapy has been delayed because of safety considerations. For example, gene therapy may provoke undesirable side effects in humans. Integration of exogenous DNA into the genome of a normal cell may cause DNA damage and possible genetic changes in the recipient cell that could possibly predispose the recipient to malignancy.

Attenuated bacteria have been used to deliver plasmids to target cells. Bacteria carrying a plasmid of interest are internalized by the target cell, the bacteria escape from the phagocytic vacuole, and lyse as the result of either metabolic attenuation (auxotrophy), an inducible autolytic mechanism, or simply from treatment with antibiotics (Sizemore et al., 1995; Courvalin et al., 1995; 1996; Darji et al., 2000; Dietrich et al., 1998; Hense et al., 2001; U.S. Patent Application 20020045587). Plasmids liberated from the bacteria are transferred into the nucleus of the host cell, leading to expression of the encoded protein. Plasmid transfer has been reported for Shigella flexneri, Salmonella typhimurium, Salmonella typhi, Listeria monocytogenes, and recombinant E. coli (reviewed in Weiss and Chakraborty, 2001). However, these techniques for gene transfer are limited by lack of cell type specificity, inefficiency, and lack of timing specificity.

Thus, additional techniques of gene therapy involving bacterial vectors, which avoid the problems set forth above, would be of significant benefit in making an efficient, safer, and high capacity gene therapy.

### SUMMARY OF THE INVENTION

The present invention provides additional compositions and methods for delivery of nucleic acids into a target cell. Furthermore, the invention provides various fusion proteins and modified bacteria that can be formulated for administration by various routes, including oral administration. Use of the compositions and methods of the invention can result in efficient and rapid delivery of one or more polynucleotides to specific tissues in a host.

Embodiments of the invention include, fusion proteins comprising a Type III secretion domain and a polynucleotide-binding domain. The Type III secretion domain can be, but is not limited to, a Shigella Type III secretion domain or a Yersinia Type III secretion domain. In a preferred embodiment, the fusion protein comprises a Type III secretion domain as set forth in SEQ ID NO:3. In other embodiments, a fusion protein includes a TAT, Antp, or HSV VP22 transduction domain with or without a secretion domain. The polynucleotide-binding domain of a fusion protein may be a deoxyribopolynucleotide (DNA) binding domain or a ribopolynucleotide (RNA) binding domain. In certain embodiments, the polynucleotide-binding domain is a sequence-specific polynucleotide-binding domain, more preferably a sequence-specific DNA binding domain. In a preferred embodiment, the sequence-specific DNA binding domain is a Lac repressor polynucleotide-binding domain. In other aspects, the sequence-specific polynucleotide-binding domain is a sequence-specific RNA binding domain.

In further embodiments, a composition may include a fusion protein as described above and a polynucleotide, wherein the polynucleotide-binding domain of the fusion protein is capable of binding to the polynucleotide. A polynucleotide of the invention may comprise one or more therapeutic or prophylactic polynucleotides, encode one or more therapeutic or prophylactic polypeptides. The therapeutic or prophylactic polynucleotide may include, but is not limted to a tumor suppressor gene, an apoptotic gene, a gene encoding an enzyme, a gene encoding an antibody, or a gene encoding a hormone. In certain aspects, the therapeutic or prophylactic gene is Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC. In further aspects, the polynucleotide may be an antisense nucleic, preferably an antisense DNA. The antisense nucleic acid or DNA may be an antisense ras, antisense myc, antisense raf, antisense erb, antisense src, antisense fms, antisense jun, antisense trk, antisense ret, antisense gsp, antisense hst, antisense bcl, or antisense abl. In still further aspects, the polynucleotide is an RNA. An RNA may be a messenger RNA, an antisense RNA, an interfering RNA or a ribozyme. In still further aspects, the polynucleotide may be a DNA-RNA hybrid. A polynucleotide of the invention may be comprised in a bacterium.

Embodiments of the invention include compositions comprising a bacterium, wherein the bacterium further comprise a fusion protein of the invention and a polynucleotide of the invention; wherein the polynucleotide binding domain of the fusion protein is capable of binding the polynucleotide. In other embodiments, a fusion protein includes, but is not limited to TAT, Antp, or HSV VP22 transduction domain with or without a seretion domain. In a preferred aspect, the bacterium is an attenuated, non-pathogenic bacterium, more preferably, the bacterium is a Shigella species, or a Yersinia species. In other aspects, the bacterium carries a mutation that causes the bacterium to undergo lysis in the gastrointestinal tract or inside the cells of the gastrointestinal tract of a subject following oral administration by the subject.

Compositions of the invention may be pharmaceutical compositions suitable for oral or parenteral delivery to a subject. In certain aspects, the composition is suitable for oral delivery to a subject.

In still further embodiments, methods of the invention include delivering one or more genes into a target cell, comprising contacting the target cell with a composition comprising a fusion protein of the invention, wherein the contacting results in delivery of the polynucleotide into the target cell. In ceratin aspects, the cell is a mammalian cell, more preferably a human cell. The human cell can be comprised in a human subject, preferably, the human subject is a patient with cancer or at risk of developing cancer.

In yet further embodiments of the invention, the methods include delivering one or more therapeutic or prophylactic polynucleotides into a target cell in a subject, comprising obtaining a composition comprising a bacterium and administering the composition to the subject. In certain aspects, the bacterium comprises a fusion protein of the invention. Administering the bacterium results in delivery of the one or more polynucleotides into a target cell of the subject. The subject can be a mammal, more preferably a human, and still more preferably a patient with cancer or a person at risk of developing cancer.

Furthermore, embodiments of the invention include methods of delivering one or more genes into a cell, comprising obtaining a bacterium capable of becoming internalized within the cell and contacting the cell with the bacterium. The bacterium may comprise a first nucleic acid encoding one or more polynucleotides; a second nucleic acid comprising an expression cassette comprising a first promoter, which is active in the cell, operably linked to a polynucleotide encoding an RNA polymerase; and a third nucleic acid comprising an expression cassette comprising a second promoter, activated by the RNA polymerase, operably linked to a polynucleotide that when expressed results in bacterium lysis. The methods further include, contacting a cell with the bacterium, which results in the internalization of the bacterium by the cell. Once internalized, the first promoter is activated resulting in the transcription of the nucleic acid encoding the RNA polymerase. The RNA polymerase in turn activates the second promoter, which results in the transcription of the nucleic acid encoding the bacterio-lytic polypeptide or peptide, resulting in the lysis of the bacterium. Lysis of the bacteria delivers one or more polynucleotides into the cell. In certain aspects, the first promoter is specific for a target cell and may become active once the bacterium is within the cell. In other aspects, the RNA polymerase is a viral RNA polymerase, preferably a T7 RNA polymerase.

In yet further embodiments, methods of the invention include delivering one or more therapeutic or prophylactic gene to a target cell in a subject, comprising obtaining a pharmaceutical composition suitable for delivery to a subject, wherein the composition comprises a bacterium capable of becoming internalized within the cell. In certain aspects, the bacteria comprise a first nucleic acid encoding one or more therapeutic or prophylactic polynucleotide; a second nucleic acid comprising an expression cassette comprising a first promoter, active in the cell, operably linked to a polynucleotide encoding an RNA polymerase; and a third nucleic acid comprising an expression cassette comprising a second promoter, activated by the RNA polymerase, operably linked to a polynucleotide that when expressed lysis the bacterium. The method may also include administering the composition to a subject, wherein the administering results in internalization of the bacterium within a target cell, activation of the first promoter, encoding of the RNA polymerase, activation of the second promoter, encoding of the lytic polynucleotide, lysis of the bacterium, and delivery of the therapeutic or prophylactic polynucleotide into the target cell.

Embodiments discussed in the context of a methods and/or composition of the invention may be employed with respect to any other method or composition described herein. Thus, an embodiment pertaining to one method or composition may be applied to other methods and compositions of the invention as well.

“A” or “an,” as used herein in the specification, may mean one or more than one. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.”

The term “about” is used to indicate that a value includes the standard deviation of error for the device or method being employed to determine the value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. illustrates a scheme for using bacteria to deliver gene therapy vectors.

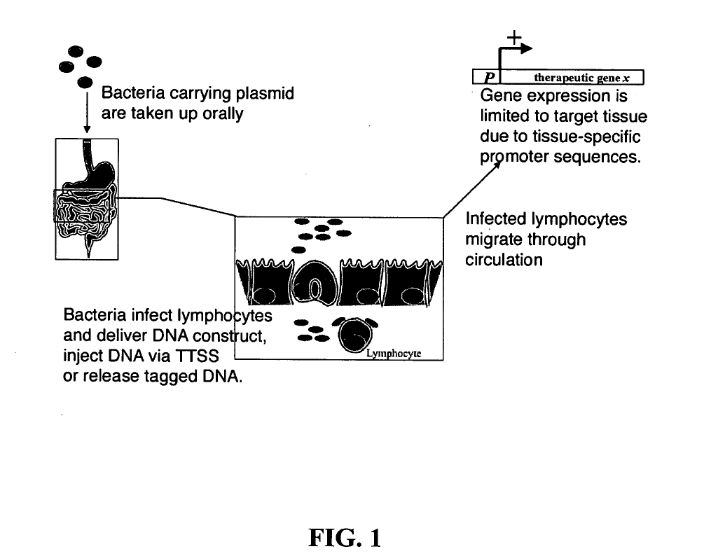

FIG. 2. illustrates a scheme for using Yersinia spp. as a gene delivery vector.

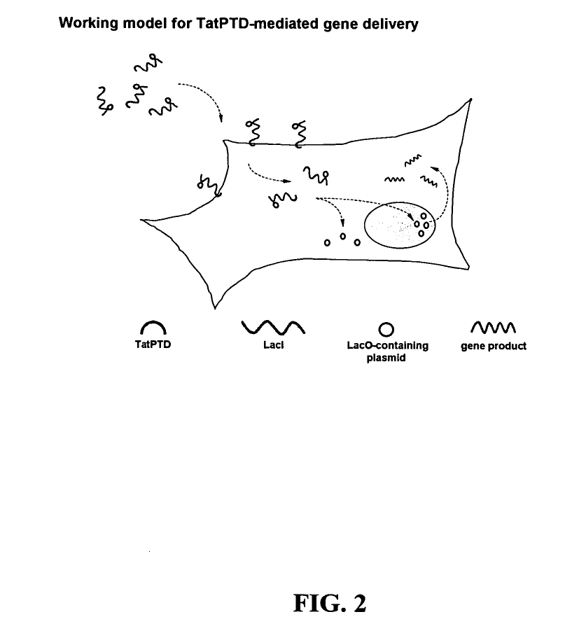

FIG. 3. illustrates a type III secretion system (TTSS).

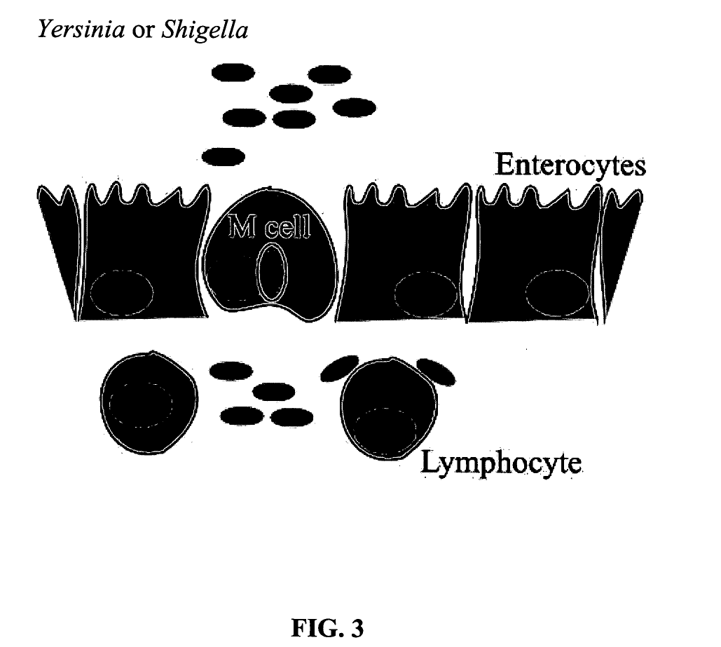

FIG. 4. illustrates the use of the TTSS of Yersinia pseudotuberculosis to deliver a GFP expressing nucleic acid into a eukaryotic cell.

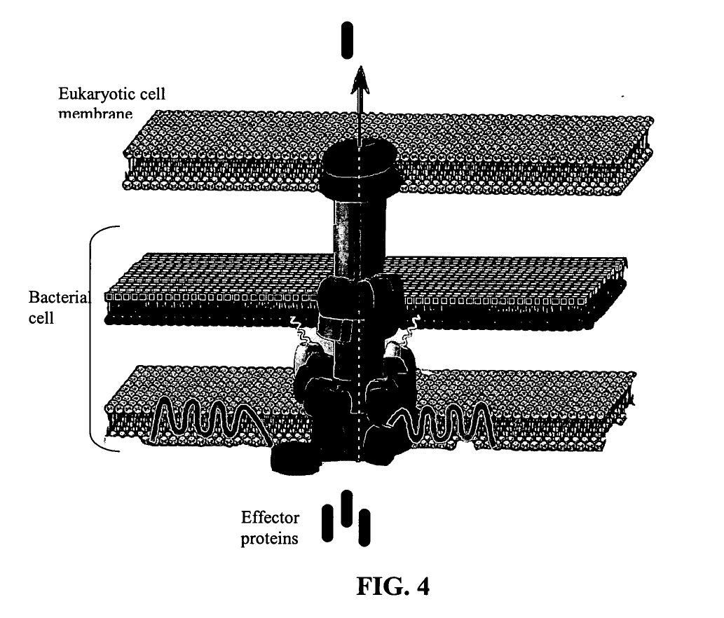

FIG. 5. illustrates a scheme for use of TAT as a carrier for DNA in gene therapy.

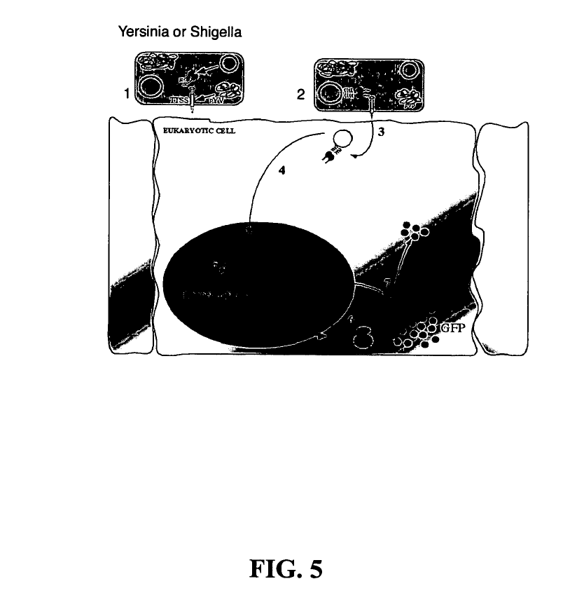

FIG. 6. illustrates exemplary fusion proteins with DNA-binding domains.

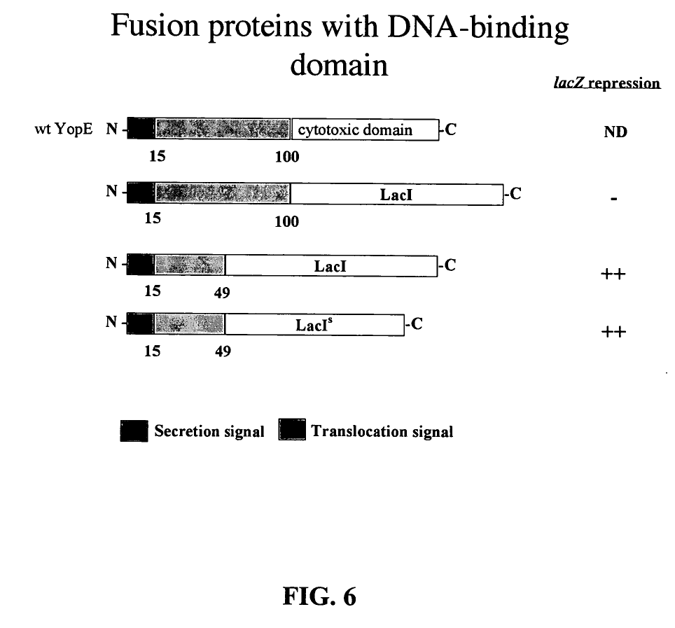

FIG. 7. shows bacterial secretion of exemplary fusion proteins.

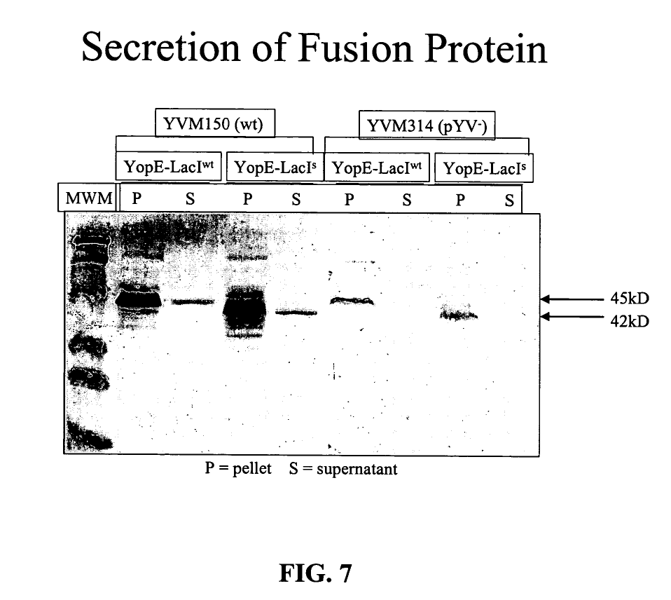

FIG. 8. shows bacterial secretion of plasmid DNA into the growth medium.

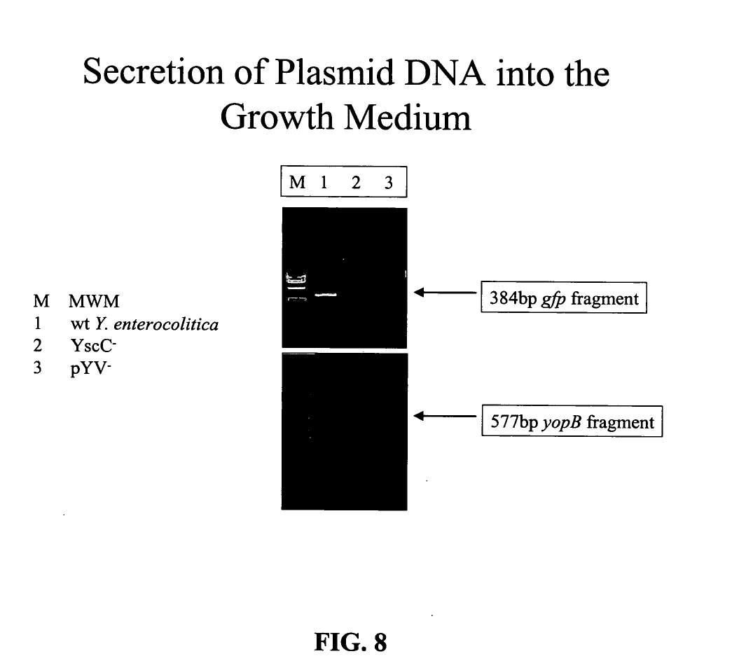

FIG. 9. shows the delivery of plasmid DNA to eukaryotic cells.

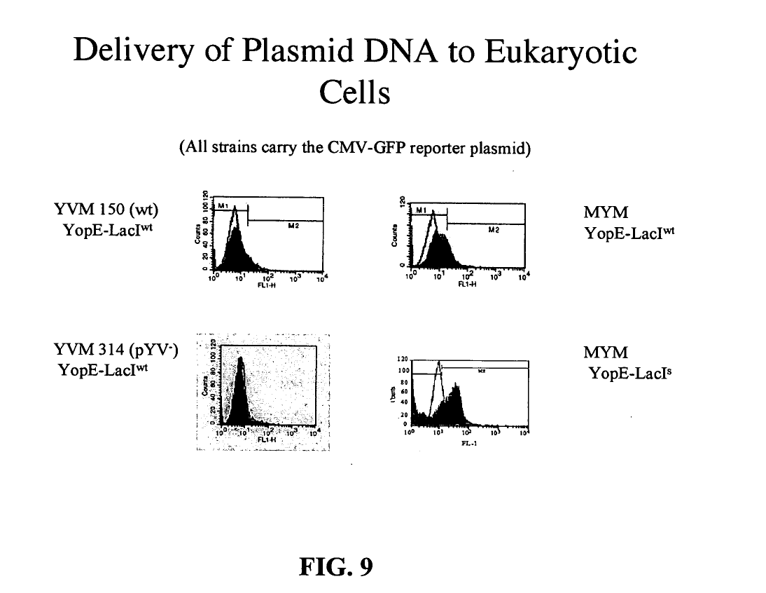

### DESCRIPTION OF ILLUSTRATIVE EMBODIMENTS

The present invention includes additional gene therapy vector compositions and methods for using such that provide efficient, specific, and high capacity polynucleotide delivery methods, vehicles, vectors, or combinations thereof. The compositions and methods of the invention also provide for the transfer of large genomic fragments facilitated by the almost unlimited coding capacity of bacterial plasmids. One use of large genomic fragments is to enable homologous recombination in the genome of a target cell. Homologous recombination may correct genetic abnormalities and mutations.

In particular, the inventors have developed various methods using modified bacterial vectors for polynucleotide delivery. For example, the methods include polynucleotide transfer from bacteria to target cell, internalization of bacterial vector by a target cell followed by polynucleotide delivery by bacterial lysis or polynucleotide secretion, bacterial delivery of polynucleotide external to a target cell by lysis or secretion, or variations and combinations thereof.

A first exemplary system exploits use of the Type III Secretion System (TTSS) of bacteria, such as Shigella and Yersinia, for delivery of a polynucleotide of interest. The TTSS is capable of injecting proteins and molecules associated with the injected proteins into cells, such as eukaryotic cells. Bacteria have engineered for injecting polynucleotides and polynucleotides associate with fusion protein into a target cell. Embodiments of the invention use novel fusion proteins that include a TTSS secretion or processing signal(s) operatively coupled to a polynucleotide binding sequence. A bacterium can be engineered to encode and express such a fusion protein. The bacterium may also include a polynucleotide encoding one or more nucleic acids, such as therapeutic or prophylactic nucleic acids. Furthermore, a fusion protein may bind or associate with a polynucleotide to be delivered by way of a polynucleotide-binding domain. When the fusion protein is internalized or secreted into a target cell it carries with it a polynucleotide of interest.

In further embodiments, a delivery method uses a bacterium that is highly efficient in becoming internalized within a target cell. Examples of such a bacterium include Shigella flexneri, which is extremely infectious and capable of entering and multiplying in host cells. The inventors have constructed a system in which the bacterium enters a target cell, lyses, and releases a polynucleotide to be delivered. In addition to including polynucleotides to be delivered to the target cell, a bacterium may contain a gene encoding an RNA polymerase under the control of a cell-specific, temporally regulated promoter. A bacterium may also include a gene that induces the lysis of the bacterium. The lytic gene may be under the control of a promoter recognized by an RNA polymerase. Internalization of the bacterium activates transcription and translation of the RNA polymerase gene and gene product. The RNA polymerase, in turn activates the transcription of a lytic gene. The bacterium lyses, releasing the polynucleotide of interest inside the target cell. By manipulating the promoters used in these methods, the inventors have developed methods of controlling cell type specificity and temporal regulation of polynucleotide release from a bacterial vehicle. This control results in decreased toxicity and increased polynucleotide delivery compared to other methods of gene delivery.

In still further embodiments, a delivery system may involve using bacterium that will release a polynucleotide outside of the host cells, for example by regulated bacterial lysis. Novel fusion proteins including a polynucleotide binding domain and a cellular uptake domain capable of facilitating intracellular delivery, such as a TAT polypeptide (for example see SEQ ID NO:4 and SEQ ID NO:7) or similar polypeptides. In addition to the fusion protein, a bacterium typically includes polynucleotides to be delivered to a target cell. The bacterium may be engineered to undergo lysis outside or inside a target cell. For example, a bacterium may contain a mutation that causes lysis following depletion of a particular nutrient(s). The polynucleotide to be delivered becomes “tagged” or associated with an uptake sequence, e.g., fusion protein. Following lysis, a target cell internalizes a fusion protein, which is associated with the polynucleotide to be delivered. Thus, regulated lysis of a bacterial vehicle, expression of protein mediators for polynucleotide delivery, and related compositions and methods of the invention can be applied to facilitate efficient and specific polynucleotide delivery to a target cell, tissue, organ or subject.

I. Bacteria as Polynucleotide Delivery Vehicles

Certain embodiments of the present invention concern compositions that include a bacterium engineered to deliver polynucleotides. In certain embodiments, a bacterium includes (1) a protein mediator of polynucleotide deliver (“protein mediator”), e.g., a fusion protein that includes a TTSS secretion or translocation domain (for example see SEQ ID NO:17), a TAT transduction domain (SEQ ID NO:4 or SEQ ID NO:7), Antp transduction domain (SEQ ID NO:8), or HSV VP22 transduction domain (SEQ ID NO:12); and (2) a polynucleotide binding domain (see for example SEQ ID NO: 14). A protein mediator of polynucleotide delivery is a protein, polypeptide, or peptide that facilitates translocation of a polynucleotide into a target cell or organelle (e.g., nucleus, mitochondria, lysosome, golgi apparatus, and/or peroxisome). A protein mediator of the invention is translocated into, within, or both into and within a target cell. An associated polynucleotide of interest is translocated along with the protein mediator and is thereby delivered to the target. In various embodiments, the protein mediator is a fusion protein comprising a translocation domain and a polynucleotide-binding domain. The polynucleotide binding sequence of a protein mediator is capable of binding to all or part of a polynucleotide of interest. Polynucleotide binding may be by general interactions with a polynucleotide or by sequence specific polynucleotide binding, such as SEQ ID NO:15.

Compositions and methods of the invention involve bacteria expressing protein mediators comprising a TTSS secretion signal or processing domain, and a polynucleotide of interest associated with the protein mediator. Typically, the bacterium will express a TTSS. Examples of such species of bacteria include Yersinia, Shigella, and Salmonella. Generally, the bacteria of the invention will be attenuated, non-pathogenic bacteria engineered to deliver of one or more polynucleotide to a cell, tissue, organ or subject. One of ordinary skill in the art is familiar with various species of bacteria that utilize TTSS.

Some embodiments of the present invention concern compositions and methods for the delivery of one or more genes into a target cell. The methods include contacting a target cell, tissue, organ, or subject with a bacterium capable of injecting or transferring a polynucleotide into, becoming internalized within, or lysing in the vicinity of a target cell. Various bacteria are contemplated for use in these methods as long as the bacteria comprise a TTSS are capable of becoming internalized within the target cell, are capable of lysing upon contacting a target cell, or are a combination thereof. In certain specific embodiments of the present invention involve delivery of one or more genes to a target cell in a subject using for example attenuated, non-pathogenic bacteria. For example, the bacteria may be a modified Shigella, Yersinia, Salmonella, or E. coli species. One of ordinary skill in the art would be familiar with the various species of bacteria that are capable of becoming internalized within a target cell and with the methods of attenuating these bacteria to render them non-pathogenic or at least reducing the pathogenicity of the bacteria.

Embodiments of the invention include bacteria engineered to lyse under particular conditions and release a polynucleotide of interest into or in proximity of a target cell. A bacterium of the invention may contain a polynucleotide that when expressed results in the lysis of the bacteria. Expression of this lytic gene may be regulated by an inducible or otherwise regulated promoter. In particular, the gene may be under the control of a promoter that is activated by a transcriptional regulator or environment. In certain embodiments, the transcriptional regulator is a RNA polymerase that specifically recognizes the promoter elements controlling expression of a lytic gene. The RNA polymerase gene itself may be under the control of a second inducible promoter. The inducible promoter may be activated under specific cellular conditions by the addition of a particular metabolite or compound, or is actived in a particular cell type or environment (hypoxic region or tissue). The promoter may also be time or location specific such that transcription of a transcriptional regulator or a lysis gene, e.g., RNA polymerase, occurs after internalization of the bacterium or upon contact with the external environs of a target cell. Bacteria of the invention may also include one or more polynucleotides encoding on or more therapeutic agents to be transferred to and/or expressed in a target cell. After internalization into or contact with a target cell, induction of a transcriptional inducer, e.g., a RNA polymerase, activates of the promoter controlling expression of the gene that causes lysis. Lysis of the bacteria causes release of the gene or genes into the target cell or in proximity of a cell.

Further embodiments include compositions and methods that utilize a bacterium that is capable of delivering a gene or genes of interest to the outside of a target cell or in proximity to a target cell. Various species of bacteria are contemplated for use in delivering polynucleotides, as long as the polynucleotide is released outside and in the vicinity of a target cell, and is taken up into the cell. For example, in specific embodiments of the present invention, the bacterium is a species of Shigella.

In yet still further embodiments, the bacterium may contain a mutation that results in bacterial cell lysis following a particular time interval, following depletion of a nutrient, following oral administration to a subject, following exposure to lysis in the gastrointestinal tract, or combinations thereof. Typically, polynucleotides are released in the extracellular space or gastrointestinal tract of a subject after bacterial lysis.

In certain embodiments of the present invention, a protein mediator may include a TAT sequence of human immunodeficiency virus (SEQ ID NO:4, 5, 6, and 7). In other embodiments, for example, a protein mediator may comprise polypeptide domains derived from an Antennapedia (Antp) protein transduction domain (SEQ ID NO:9 and 10), (SEQ ID NO:8) or a polypeptide from the HSV-1 structural protein VP-22 (SEQ ID NO:11 and 12). These “tags” or protein mediator domains are discussed below.

A. Type III Secretion System (TTSS)

The type III secretion system (TTSS) is class of specialized protein secretion systems that deliver bacterial virulence proteins directly into a host cell (reviewed in He, 1998). Bacterial pathogens that utilize this system are those known to be responsible for a number of diseases in plants, animals, and humans, such as rice leaf blight and diarrhea. These pathogens are very diverse in their taxonomy, host range, and related disease symptoms. A unique feature of the TTSS is the ability to deliver bacterial virulence proteins directly into host cells (Rosqvist et al., 1994). This protein delivery mechanism enables bacterial pathogens to gain access to a vast number of host targets, which would have been inaccessible if bacterial virulence proteins were delivered only to the surface of host cells.

Molecules destined to travel through the TTSS pathway are targeted to the secretion organelle by information contained within the first 120 amino acids of a protein or an associated protein (Cheng et al., 1997; Michiels and Cornelis, 1991; Sory et al., 1995). This secretion and translocation domain is not cleaved upon secretion, and when added to the amino terminus of reporter proteins, is capable of delivering them into host cells (Sory and Cornelis, 1994). In the bacterial cytoplasm, this domain serves as the binding site for a family of related customized chaperones, which are not secreted and are released upon secretion of the cognate effector proteins (Page and Parsot, 2002; Schesser et al., 1996; Wattiau et al., 1994; Wattiau and Cornelis, 1993). Recent crystallographic studies have shown that these chaperones maintain significant portions of the amino terminus of the effector proteins in an extended conformation that is presumably primed for rapid secretion (Birtalan et al., 2002; Stebbins and Galán, 2001). Furthermore, the crystal structure of two of these effectors, SptP and YopE, in complex with their respective chaperones, revealed that the main chain path across the chaperones is strikingly similar, despite the notable lack of overall primary sequence similarity among chaperone binding domains (Birtalan et al., 2002; Stebbins and Galán, 2001). The nucleic acid sequence and protein sequence of YopE is provided in SEQ ID NO: 1 and 2, respectively. This feature may serve as recognition signal for targeting the complexes to the TTSS.

Bacteria use TTSS for difference purposes. For example, the TTSS of plant pathogenic bacteria is used to secrete virulence proteins that cause leakage of plant nutrients to the extracellular space of infected tissues (Sigee and AL-Rabaee, 1986). Intracellular pathogens such as Salmonella spp. and Shigella spp. use TTSS for invasion of host cells (Galan and Bliska, 1996; Menard et al, 1996). Yersinia spp. also invade host cells, but the TTSS is not used for invasion. Yersinia spp. TTS resist uptake of bacteria by both phagocytes and non-phagocytic cells in the later stages of pathogenesis when the bacteria are extracellular (Simonet et al., 1992).

Clusters of genes associated with TTSS are either in extrachromosomal elements such as plasmids or in specific chromosomal regions. Analysis of isolated gene clusters of many apparently diverse bacteria reveal unexpected sequence similarities among themselves and to flagellar assembly genes (He, 1998). A common function of these broadly conserved genes is secretion of proteins and their sequences (with few exceptions) are not related to genes involved in any other protein secretion systems (Salmond and Reeves, 1993).

II. Polypeptides Related to Polynucleotide Delivery

Embodiments of the present invention generally pertain to protein mediators of polynucleotide translocation. Protein mediators include peptides, polypeptides, or fusion proteins comprising a protein translocation domain, (e.g., a TTSS secretion domain) and a polynucleotide binding domain for the delivery of a polynucleotide to the interior or exterior of a target cell. Further embodiments pertain to fusion proteins that include protein translocation domains such as TAT (SEQ ID NO:4 or 7), Antp (SEQ ID NO:8), or HSV VP22 (SEQ ID NO:11) polypeptide coupled with a polynucleotide binding domain, such as SEQ ID NO:15.

A. TTSS Secretion or Translocation Domain

In certain embodiments of the invention, a fusion protein includes a TTSS secretion or translocation domain. Throughout this application, the term “TTSS secretion domain” refers to any portion of a polypeptide that signals or enhances secretion or translocation through the TTSS. A TTSS secretion domain may be derived from any species of bacteria having a TTSS. Exemplary amino acid sequence of selected TTSS secreted polypeptides includes SEQ ID NO:3.

As used herein, a TTSS secretion domain refers to a polypeptide sequence that is a substrate for the TTSS of an appropriate species or engineered bacteria. The bacteria can be any species of bacteria that is known to one of ordinary skill in the art to utilize a TTSS. For example, the species of bacteria may be Pseudomonas spp., Yersinia spp. or Shigella spp. As discussed above, many proteins have been identified that are associated with the TTSS, and a TTSS secretion domain can be a polypeptide sequence from any such protein, as long as the domain is capable of facilitating the secretion and/or uptake of a polypeptide into a target cell. One of ordinary skill in the art would be familiar with the many TTSS polypeptide sequences that are available for use in the context of the fusion proteins of this invention.

In certain embodiments of the invention, the fusion protein includes a protein transduction domain that includes, but is not limited to a TAT polypeptide (SEQ ID NO:4, 6 or 7) (Nagahara et al., 1998, herein specifically incorporated by reference in its entirety).

In further embodiments, the polypeptide capable of facilitating uptake of the fusion protein into a target cell is a polypeptide that functions in a manner similar to a TAT polypeptide to promote uptake of the fusion protein into a cell. For example, fusion protein uptake is facilitated by a polypeptide from an Antp protein transduction domain (SEQ ID NO:8) (Derossi et al., 1994; herein specifically incorporated by reference in its entirety). The polypeptide capable of facilitating uptake of the fusion protein into a target cell may also be a polypeptide from the HSV-1 structural protein VP22 (SEQ ID NO:11) (Elliott and O'Hare, 1997; herein specifically incorporated by reference in its entirety). One of ordinary skill in the art is familiar with these polypeptide sequences.

In certain embodiments, a variety of protein transduction domains (PTDs) may be included in fusion proteins of the invention. To date, sequence analysis of a number of TTSS gene clusters is available. For example, He (1998) (herein specifically incorporated by reference in its entirety) lists published proteins that are encoded by genes in various TTSS gene clusters, and also discusses in detail the TTSS of Yersinia spp., Salmonella spp., and Shigella spp.

Salmonella and Shigella use TTSS to invade host cells. Both Shigella and Salmonella secrete multiple proteins, four of these proteins are well characterized: IpaA, IpaB, IpaC, and IpaD in Shigella (Menard et al., 1993), and their functional homologs SipA, SipB, SipC, and SipD in Salmonella (Kaniga et al., 1995A, Kaniga et al., 1995B). IpaB-D and SipB-D are required for the entry of Shigella and Salmonella, respectively, into cultured epithelial cells. The IpaB and IpaC proteins assemble into multiprotein complexes that appear to be essential for their function.

Other bacterial proteins that are used to facilitate uptake of bacterial proteins by target cells include TAT protein transduction domains (SEQ ID NO:4 and 7) and Antp protein transduction domain (SEQ ID NO:8), as well as the HSV-1 structural protein VP22. TAT and Antp protein transduction domains promote endocytosis of proteins upon binding to target cell surface glycosaminoglycans (Console et al., 2003). PTDs are short basic peptide sequences present in many cellular and viral proteins that mediate translocation across cellular membranes. These cell-permeable peptides are functional when fused to recombinant polypeptides or when chemically coupled to their cargo. The mechanism responsible for PTD-mediated membrane translocation is controversial and may vary among the various PTDs reported in the literature. Thus direct physical interaction with membrane lipids resulting in vector delivery to cells has been proposed for the Antp PTD, whereas uptake by TAT PTD seems to require the expression of glycosaminoglycans on the cell surface.

The exploitation of bacterial TTSS to efficiently deliver high capacity polynucleotides into target cells is novel. There have been limited reports attempting to use TAT, Antp, and HSV VP22 polypeptides in gene delivery (Nakanishi et al., 2003; U.S. Pat. No. 6,376,248). However, these techniques lack target cell specificity, are inefficient, and lack timing specificity.

B. Polynucleotide Binding Domains

A protein mediator of the invention, e.g., a fusion protein, may comprise a polynucleotide-binding domain, such as LacI (SEQ ID NO:14). The fusion proteins are also used in compositions and methods of the invention for delivery one or more polynucleotide into a target organelle, cell, tissue, organ or subject.

Various polynucleotide-binding domains are known to those of ordinary skill in the art and may be engineered into protein mediators of the invention. In certain embodiments, the polynucleotide-binding domain is a DNA binding domain, an RNA binding domain, or a combination thereof. One of ordinary skill in the art is familiar with the various binding domains that are available for application in the present invention. In certain embodiments of the present invention, the polynucleotide binding domain of a fusion protein is a sequence-specific polynucleotide binding sequence, such as LacI (SEQ ID NO:14, which is encoded by SEQ ID NO:13), which binds a LacO sequence (exemplified in SEQ ID NO:15).

The sequence-specific polynucleotide-binding domain of a fusion protein may be a sequence-specific DNA binding sequence, or a sequence-specific RNA binding sequence. For example, the sequence-specific DNA binding sequence may be all or part of a Lac repressor polypeptide (SEQ ID NO:14) that specifically bindings to Lac operator (SEQ ID NO:15).

C. Fusion proteins

Protein mediators of polynucleotide deliver include fusion proteins. The term “fusion protein” refers to a protein formed by the joining of different peptide, polypeptide, or protein segments by genetic or chemical methods wherein the joined ends of the peptide, polypeptide, or protein segments may be directly adjacent to each other or may be separated by linker or spacer moieties such as amino acid residues or other linking groups. Fusion proteins of the invention are non-naturally occurring proteins, wherein the domains of the fusion protein may be derived from one or more proteins or artificial molecules.

One of ordinary skill in the art would be familiar with methods for preparing fusion proteins. The fusion protein may be constructed by a variety of mechanisms including, but not limited to, standard DNA manipulation techniques and chemical assembly via subunit parts of the fusion protein. The chemical assembly may lead to an equivalent of the fusion protein. In certain embodiments, the fusion protein is produced by standard recombinant DNA techniques. Polynucleotides encoding fusion proteins of the invention may be stably integrated into the genome of or maintained episomally within a bacterial host.

The fusion proteins of the present invention, as exemplified in SEQ ID NO:17, include a polypeptide domain capable of facilitating uptake of the fusion protein into a target cell. The term “polypeptide,” as used herein, refers to any amino acid or amino acid-like sequence having consecutive amino acids or amino acid like monomers. Fusion protein includes consecutive amino acid sequences forming a protein translocation domain, e.g., a TTSS secretion domain, a protein transduction domain, a polynucleotide binding domain, or combinations thereof. For example, a fusion protein or a peptide component of a fusion protein can include, but is not limited to, about 10, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid or amino acid-like residues of a fusion protein, and any range therein.

“Peptide” refers to any amino acid or amino acid-like sequence that includes 100 or fewer consecutive amino acid sequence of the amino acid sequence of the protein. “Peptide” includes consecutive amino acid sequences from a bacterial or human, or from any other species, such as mouse. Thus, for example, a particular peptide may include 1, 2, 3, 4, 5, 6, 7, 8, 9, 10, 20, 30, 40, 50, 60, 70, 80, 90 or 1000 consecutive amino acids or ranges therebetween. Additional amino acids can also be included, which may be amino acid sequences that are not associated with a particular protein. The term polypeptide is used generally to include polymers of amino acids and amino acid like molecules of various sizes and includes peptides and proteins.

The polypeptide domain capable of facilitating translocation or uptake of a fusion protein into a cell include TTSS secretion domains, TAT protein transduction domains (SEQ ID NO:4 and 7), Antp protein transduction domain (SEQ ID NO:8), and VP22 protein transduction domain (SEQ ID NO:12) as described herein. Included in the definition of these particular polypeptides or polypeptide domains are polypeptide equivalents. It is well understood that inherent in the definition of a “polypeptide equivalent” is the concept that there is a limit to the number of changes that may be made within a defined portion of the molecule and still result in a molecule with an acceptable level of equivalent biological activity, e.g., ability of the polypeptide to facilitate uptake of fusion protein into a target cell. “Polypeptide equivalent” is thus defined herein as any polypeptide in which some, or most, of the amino acids may be substituted so long as the polypeptide retains substantially similar activity in the context of polynucleotide delivery. Of course, a plurality of distinct proteins/polypeptides/peptides with different substitutions may easily be made and used in accordance with the invention.

Additionally, in the context of the invention, a polypeptide equivalent can be a homologous polypeptide from any species or organism, including, but not limited to, a bacteria. One of ordinary skill in the art will understand that many polypeptide equivalents would likely exist and can be identified using commonly available techniques. Of course, any homologous polypeptide may be substituted in some, even most, amino acids and still be a “polypeptide equivalent,” so long as the polypeptide retains substantially similar activity in the context of the uses set forth.

These amino acid sequences may, for example, have an amino acid identity of about 40% with a known polypeptide domain (e.g., a TAT polypeptide), and a chemical identity (presence of identical or chemically similar amino acids) of about 60-70%, indicating that they are biologically equivalent polypeptides to the known polypeptide. Therefore, polypeptides such as these would be polypeptide equivalents because only certain amino acids are substituted when compared with a known polypeptide.

The present invention may utilize polypeptides or polypeptide equivalents purified from a natural source or from recombinant material. Those of ordinary skill in the art would know how to produce these polypeptides using recombinant methods. This material may use the 20 common amino acids in naturally synthesized proteins, or one or more modified or unusual amino acids.

Generally, “purified” will refer to a composition that has been subjected to fractionation to remove various other proteins, polypeptides, or peptides, and which composition substantially retains its activity. Purification may be substantial, in which the desired polypeptide or equivalent is the predominant species, or to homogeneity, which purification level would permit accurate degradative sequencing.

Amino acid sequence mutants of known polypeptides, such as TTSS secreted polypeptides, TAT, Antp, and VP22 sequences are encompassed within the definition of “polypeptide equivalent.” Amino acid sequence mutants of the polypeptide can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide.

Amino acid substitutions are generally based on the relative similarity of the amino acid side-chain substituents, for example, hydrophobicity, hydrophilicity, charge, size, and the like. An analysis of the size, shape and type of the amino acid side-chain substituents reveals that arginine, lysine and histidine are all positively charged residues; that alanine, glycine, and serine are all a similar size; and that phenylalanine, tryptophan, and tyrosine all have a generally similar shape. Therefore, based upon these considerations, arginine, lysine, and histidine; alanine, glycine, and serine; and phenylalanine, tryptophan, and tyrosine; are defined herein as biologically functional equivalents. In making changes, the hydropathic index of amino acids may be considered. The importance of the hydropathic amino acid index in conferring interactive biological function on a protein is generally understood in the art (Kyte and Doolittle, 1982, incorporated by reference herein). It is understood that an amino acid can be substituted for another having a similar hydrophilicity value and still obtain a biologically equivalent protein, as detailed in U.S. Pat. No. 4,554,101, which is incorporated herein by reference in its entirety.

As set forth above, the fusion proteins of the present invention typically include a TTSS secretion or processing domain, a TAT protein transduction domain (SEQ ID NO:4 and 7), an Antp protein transduction domain (SEQ ID NO:8), an HSV VP22 transduction domain (SEQ ID NO:12) or a derivative thereof fused with a polynucleotide binding domain, such as SEQ ID NO: 14.

The term “a sequence essentially as set forth” means that the sequence substantially corresponds to a portion of a particular sequence and has relatively few amino acids that are not identical to, or is a biological functional equivalent of, the amino acids of referenced sequence.

III. Nucleic Acids Related to Polynucleotide Delivery

Various aspects of the present invention pertain to nucleic acid compositions, e.g., polynucleotides to be delivered or polynucleotides encoding engineered polypeptides. Nucleic acid compositions may include polynucleotides encoding fusion proteins of the invention, therapeutic polypeptides, or prophylactic polynucleotides. Embodiments include compositions and methods for nucleic acid transfer, including polynucleotides or a bacterium comprising one or more polynucleotides, such as one or more therapeutic polynucleotide, prophylactic polynucleotide, nucleic acid encoding a polypeptide engineered to deliver a nucleic acid or a combination thereof.

As used in this application, the term “polynucleotide” refers to a nucleic acid molecule, RNA or DNA, that has been isolated free of total genomic DNA or RNA. Therefore, a “nucleic acid encoding a therapeutic or prophylactic polynucleotide” refers to a nucleic acid segment that contains the coding sequences of a therapeutic or prophylactic polypeptide, nucleic acid, or genomic segment, yet is isolated away from, or purified and free of, total genomic DNA and proteins. In certain embodiments, a polynucleotide of the invention need not encode a therapeutic nucleic acid, but may encode a portion of a genome that may be used to correct detrimental sequence present in a subjects genome, such as replacing a defective splice junction, promoter region, or repetitive element, and the like.

A nucleic acid contemplated for inclusion in the compositions and methods of the present invention includes, but is not limited to a plasmid, an artificial chromosome, a genomic fragment, an expression vector, or an expression cassette. A nucleic acid may or may not include one or more polynucleotide regions encoding, for example, an entire protein sequence, a functional protein domain of a particular protein, a polypeptide of a particular protein, a polypeptide equivalent or may encode a functional portion of the genome that may or may not be transcribed or translated. In certain embodiments, the polynucleotide may be derived from genomic DNA, i.e., cloned directly from the genome of a particular organism.

In other embodiments, a polynucleotide may be complementary DNA (cDNA). The term “cDNA” is intended to refer to DNA derived from a RNA template. The advantage of using a cDNA, as opposed to genomic DNA or an RNA transcript is stability and the ability to manipulate the sequence using recombinant DNA technology (See Sambrook, 2001; Ausubel, 1996). Alternatively, cDNAs may be advantageous because it represents coding regions of a polypeptide and eliminates introns and other regulatory regions. In other embodiments, a polynucleotide may be produced synthetically.

It may be advantageous to combine portions of a genomic DNA segment with a cDNA or synthetic sequences to generate specific constructs. There may be times when a full or partial genomic sequence is used. For example, where an intron is desired in the ultimate construct, a genomic clone will need to be used. Introns may be derived from genes known to those of ordinary skill in the art.

The term “gene” is used for simplicity to refer to a polynucleotide that encodes a functional protein, polypeptide, or peptide. As will be understood by those in the art, this functional term includes genomic sequences, cDNA sequences, and smaller engineered gene segments that express, or may be adapted to express, proteins, polypeptides, domains, peptides, fusion proteins, and mutants. Any gene is contemplated for inclusion in the invention. A person of ordinary skill in the art would understand that commonly available experimental techniques can be used to identify or synthesize polynucleotides encoding any gene. The present invention also encompasses chemically synthesized mutants of particular genes.

In some embodiments, the polynucleotide further comprises a polypeptide binding sequence. As used herein, “polypeptide binding sequence” references to any polynucleotide sequence capable of preferential binding to a corresponding polypeptide domain. One example of a polypeptide binding sequence is the Lac operator, which is capable of binding a Lac repressor polypeptide domain.

Various polynucleotides of the invention may be isolated substantially away from other sequences. “Isolated substantially away from other sequences” means that the nucleic acid of interest forms part of the polynucleotide, and that the segment does not contain large portions of other naturally occurring nucleic acids, such as large chromosomal fragments or other functional genes or cDNA coding regions. Of course, this refers to the nucleic acid segment as originally isolated, and does not exclude genes, polynucleotides or coding regions later added to the segment by human manipulation.

The term “biological functional equivalent” is well understood in the art and is further defined as sequences that encode a polypeptide of about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of sequence will be sequences that are “essentially as set forth” in the respective sequence provided the biological activity of the protein is maintained. The term “essentially as set forth in” in a referenced nucleic acid sequence is used in the same sense as described above and means that the nucleic acid sequence substantially corresponds to a portion of a referenced sequence.

In certain embodiments, one may wish to employ antisense constructs, which include other elements, for example, those which include C-5 propyne pyrimidines. Oligonucleotides that contain C-5 propyne analogues of uridine and cytidine have been shown to bind RNA with high affinity and to be potent antisense inhibitors of gene expression (Wagner et al., 1993).

As an alternative to targeted antisense delivery, a polynucleotide may be a targeted ribozyme. The term “ribozyme” refers to an RNA-based enzyme capable of targeting and cleaving particular base sequences in both DNA and RNA. Ribozymes can either be targeted directly to cells, in the form of RNA oligonucleotides incorporating ribozyme sequences, or introduced into the cell as an expression vector encoding the desired ribozymal RNA. Ribozymes may be used, applied, and modified in much the same way as an antisense polynucleotide. For example, one could incorporate non-Watson-Crick bases, or make mixed RNA/DNA oligonucleotides, or modify the phosphodiester backbone.

Alternatively, the antisense oligo- and polynucleotides according to the present invention may be provided as mRNA via transcription from expression cassette that carry nucleic acids encoding the oligo- or polynucleotides. Expression cassettes are described in greater detail below.

The polynucleotides utilized in the present invention may also be interfering RNA. Interfering RNA refers to the ability of exogenous double strand RNA to suppress the expression of the gene that corresponds to the double strand RNA sequence.

A. Therapeutic and Prophylactic Polynucleotides

Bacterial vectors of the invention may be used to deliver various nucleic acids encoding therapeutic or prophylactic nucleic acids into or in the proximity of a target cell. In certain embodiments, the nucleic acids include one or more therapeutic or prophylactic polynucleotides. Various polynucleotides encode a functional protein, polypeptide, or peptide. “Therapeutic polynucleotide” is a polynucleotide that can be administered to a subject for treating or ameliorating a disease. “Prophylactic polynucleotide” is a polynucleotide that can be administered to a subject for preventing a disease.

The therapeutic or prophylactic polynucleotide may be a tumor suppressor gene (or encoding a tumor suppressor), a pro-apoptotic gene, or a gene encoding a hormone, antibody, or enzyme. Examples of therapeutic and prophylactic genes include, but are not limited to Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, and MCC.

Other examples of therapeutic or prophylactic genes include genes encoding enzymes, which include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, and a reporter gene.

Further examples of therapeutic or prophylactic polynucleotides include the nucleic acids encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, and human thymidine kinase.

Therapeutic polynucleotides also include genes encoding hormones. Examples include, but are not limited to, polynucleotides encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

B. Expression Cassettes and Promoters

Certain embodiments include compositions and methods involving a polynucleotide wherein the polynucleotide is comprised in an expression cassette. For example, specific aspects of the present invention pertain to methods of delivering one or more genes into a cell that involve contacting the cell with a bacterium containing polynucleotides comprised in expression cassettes.

Throughout this application, the term “expression cassette” is meant to include any type of genetic construct containing a polynucleotide in which all or part of the polynucleotide sequence is capable of being transcribed. The polynucleotide may include one or more polypeptide coding regions. The transcript may be translated into a protein or polypeptide, but it need not be. Thus, in certain embodiments, expression includes both transcription of a polynucleotide and translation of a mRNA into a polypeptide.

One of skill in the art would understand the techniques relating to use of expression cassettes to deliver polynucleotide sequences to cells or subjects. Particular aspects of these techniques of these techniques are summarized in this specification. This brief summary is in no way designed to be an exhaustive overview of all available experimental techniques related to expression cassettes since one of skill in the art would already be familiar with these techniques.

In order for the expression cassette to effect expression of a polypeptide, the polynucleotide may be under the transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrase “operatively linked” means that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence. One of skill in the art would understand how to use a promoter or enhancer to promote expression of a particular polynucleotide.

In certain embodiments of the present invention, a particular polynucleotide, such as a polynucleotide encoding RNA polymerase, is under the control of a promoter that is cell-type specific. Specifically, such a promoter is only active when it is located within a cell of a particular type. Certain promoters used to control expression of a polynucleotide may also be timing-specific in that the promoter becomes active following exposure to a certain environment, such as location within a specific cell type.

In certain embodiments, a polynucleotide may be under the control of a promoter that is capable of binding RNA polymerase. For example, the polynucleotide may be a polynucleotide in a bacterium that contains a lysis gene under the control of such a promoter. The promoter may be capable of binding a specific RNA polymerase, such as viral RNA polymerase (for example, T7 RNA polymerase). Utilization of specific promoters in association with the present invention allows for both cell type and timing specificity of polynucleotide expression.

A regulatable promoter may be used which may be inserted as part of a DNA fragment which additionally carries a polynucleotide of interest. However, other promoter systems inserted in the plasmids in accordance with the principles of the invention and influenced by other external factors such as temperature and promoters which are inducible with chemicals or regulatable by means of metabolites, such as lac, trp and deo promoters. In certain embodiments, promoters are regulated by antibiotics or derivatives thereof, including, but not limited to tetracycline and its analogs.

In certain embodiments, promoters that are specifically expressed in the intracellular environment to drive the synthesis of T7 polymerase or other specific polymerases will be used. Non-limiting examples of intracellular promoters are those for uhpT, bioA, lysA, fhuA, sitA, sufA, pstS, and phoA.

In certain embodiments of the invention, the delivery of an expression cassette in a cell may be identified in vitro or in vivo by including a marker in the expression vector. The marker would result in an identifiable change to the transfected cell permitting easy identification of expression. The selectable marker employed is not believed to be important, so long as it is capable of being expressed along with the polynucleotide of the expression cassette. Examples of selectable markers are well known to one of skill in the art.

In certain embodiments of the invention, the use of internal ribosome entry sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1988). One of skill in the art would be familiar with use of IRES in expression cassettes.

In expression, one will typically include a polyadenylation signal to effect proper polyadenylation of the transcript. The nature of the polyadenylation signal is not believed to be crucial to the successful practice of the invention, and/or any such sequence may be employed. One of skill in the art would understand how to use these signals to effect proper polyadenylation of the transcript.

In certain embodiments of the present invention, the expression cassette is comprised in a bacterium or a bacterial vector that may or may not be able to become internalized within a target cell.

In certain embodiments of the invention, a targeted cell may be identified in vitro or in vivo by including a marker in the expression vector. Such markers would confer an identifiable change to the cell permitting easy identification of cells containing the expression vector. A positive selectable marker is one in which the presence of the marker allows for its selection, while a negative selectable marker is one in which its presence prevents its selection. An example of a positive selectable marker is a drug resistance marker. Examples of selectable and screenable markers are well known to one of skill in the art.

IV. Diseases for Treatment and Prophylaxis

The present invention contemplates methods of delivering one or more therapeutic or prophylactic polynucleotide to a subject. The subject can be a mammal, such as a human. The subject may be disease-free, and delivery of one or more polynucleotide may be for preventing onset of a disease in the subject. Delivery of one or more polynucleotide to such a subject can be for preventing a disease or condition for which such preventive treatment by delivery of one or more prophylactic polynucleotide is deemed beneficial by one of skill in the art.

Alternatively, treatment with one or more polynucleotide may be deemed beneficial by one of skill in the art to a subject already affected by a disease. For example, in some embodiments of the present invention, the subject may be a patient with a disease associated with abnormal cell proliferation, such as cancer. Treatment or prevention of any type of cancer by delivery of one or more therapeutic polynucleotide is contemplated by the methods of the present invention. For example, a cancer may be breast cancer, lung cancer, prostate cancer, ovarian cancer, brain cancer, liver cancer, prostate cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, or leukemia.

In other embodiments of the present invention, the subject is afflicted by other conditions. For example, the condition may be a hormonal defect, an infection, or an enzyme deficiency.

Polynucleotides delivered for the purpose of preventing or treating a disease include, by way of example, encode tumor suppressors, inducers apoptosis, enzymes, antibodies, or hormones.

In particular embodiments of the present invention, delivery of the one or more therapeutic or prophylactic polynucleotide is targeted to cells of a specific type in a subject. Use of specific promoters, as discussed above, facilitates such targeting.

V. Combination Therapies

Embodiments of the invention provide for methods of delivering one or more therapeutic or prophylactic polynucleotides to a subject in combination with a second treatment. For example, in certain embodiments, the subject is a patient with cancer. A variety of other therapies are known to one of skill in the art and may be used in combination with the compositions and methods of the invention.

In the case of cancer, examples of some of the existing cancer therapies include radiation therapy, chemotherapy, surgical therapy, immunotherapy, and other gene therapies. Examples of other cancer therapies include phototherapy, cryotherapy, toxin therapy, or hormonal therapy. One of skill in the art would know that this list is not exhaustive of the types of treatment modalities available for cancer and other hyperplastic lesions.

In order to increase the effectiveness of delivering one or more therapeutic or prophylactic polynucleotides to a target cell of interest, it may be desirable to combine these compositions with other agents effective in the treatment of the disease of interest. These compositions would be provided in a combined amount effective to kill or inhibit proliferation of the cell. This process may involve contacting the cells with the therapeutic or prophylactic preparation of the present invention and the agent(s) or second factor(s) at the same time. This may be achieved by contacting the cell with a single composition or pharmacological formulation that includes both agents, or by contacting the cell with two distinct compositions or formulations, at the same time, wherein one composition includes the polynucleotide(s) to be delivered and an additional therapeutic agent.

Alternatively, the polynucleotide therapy may precede or follow a second agent or treatment by intervals ranging from minutes to weeks. In embodiments where a second agent and polynucleotide therapy are applied separately, one would generally ensure that a significant period of time did not expire between the time of each delivery. In such instances, it is contemplated that one may contact the cell with both modalities within about 12-24 h of each other and, more preferably, within about 6-12 h of each other. In some situations, it may be desirable to extend the time period for treatment significantly, however, where several days (d) (2, 3, 4, 5, 6 or 7) to several weeks (wk) (1, 2, 3, 4, 5, 6, 7 or 8) lapse between the respective administrations.

Administration of bacteria and bacterial vectors of the present invention to a patient will follow general protocols for the administration of chemotherapeutics, taking into account the toxicity, if any. It is expected that the treatment cycles would be repeated as necessary. It also is contemplated that various standard therapies, as well as surgical intervention, may be applied in combination with the described therapy.

VI. Pharmaceutical Compositions

Certain embodiments, include compositions of polypeptides and polynucleotides bound thereto, wherein the composition is a pharmaceutically acceptable composition. The composition may be suitable for oral or parenteral delivery to a subject. In a specific embodiment, the composition is suitable for oral delivery to a subject.

Certain other embodiments of the present invention pertain to methods of delivery of one or more polynucleotide to a target cell, such as a target cell in a subject, that involve pharmaceutical preparations of polynucleotides comprised in a bacterium.

The phrases “pharmaceutical,” “pharmaceutically,” or “pharmacologically acceptable” refer to molecular entities and compositions that do not produce an adverse, allergic or other untoward reaction when administered to an animal, or human, as appropriate. As used herein, “pharmaceutical” includes any and all solvents, dispersion media, coatings, antibacterial and antifungal agents, isotonic and absorption delaying agents and the like. The use of such media and agents for pharmaceutically active substances is well known in the art.

As to the meaning of “pharmaceutical” in the context of bacterial vectors, the composition includes one or more bacterium suitable for oral or parenteral delivery to a subject. In various embodiments, the bacteria are attenuated or non-pathogenic. In this context, “pharmaceutical” also pertains to any such media and additives required to maintain viability of the bacterium. One of ordinary skill in the art would be familiar with agents that can be added to the compositions that can be added without producing any adverse, allergic, or other untoward reaction in the recipient subject.

Except insofar as any conventional media or agent is incompatible with the bacteria, bacterial vectors, or active ingredients, its use in the therapeutic compositions is contemplated. Supplementary active ingredients, such as one or more additional therapeutic or prophylactic agents, can also be incorporated into the compositions. One of ordinary skill in the art would be familiar with methods and techniques used to formulate pharmaceutical compositions to include additional supplementary active ingredients.

Certain embodiments include compositions suitable for oral administration. The term “oral administration” includes any form of administration of therapeutic substance through the oral cavity and into the gastrointestinal tract. The pharmaceutical compositions of the present invention may be formulated for oral delivery to a subject. For example, the compositions may be formulated for oral administration through a nasogastric tube, through solutions that can be swallowed, and the like.

Certain embodiments of the present invention include pharmaceutical compositions that are suitable for parenteral administration, e.g., formulated for injection by any route other than through the digestive tract, such as intravenous, intramuscular, subcutaneous, or even intraperitoneal routes. Typically, the compositions can be prepared as injectables, either as liquid solutions or suspensions; solid forms suitable for using to prepare solutions or suspensions upon the addition of a liquid prior to injection can also be prepared; and the preparations can also be emulsified. For those compositions that include a bacterium, appropriate additives can be included to maintain viability of the bacterium upon administration. Pharmaceutical preparations of the present invention can be formulated for oral or parenteral delivery by any method known to those of ordinary skill in the art.

Pharmaceutical preparations that include bacteria may include appropriately buffered media, nutrients, and other additives to maintain viability of the bacteria. One of ordinary skill in the art would be familiar with methods to formulate bacteria for oral or parenteral administration, and appropriate pharmaceutical additives that can be safely included in the preparations.

The carrier of the pharmaceutical preparations of the present invention may include a solvent or dispersion medium containing, for example, water, ethanol, polyol (for example, glycerol, propylene glycol, and liquid polyethylene glycol, and the like), suitable mixtures thereof, and vegetable oils.

The proper fluidity can be maintained, for example, by the maintenance of the required particle size in the case of dispersion and by the use of surfactants. Compositions of the present invention that do not include bacteria may include various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, gelatin.

Injectable solutions are prepared by incorporating the active compounds or bacteria in the required amount in the appropriate solvent with various other ingredients enumerated above, as required. If the pharmaceutical preparation does not include bacteria, filtered sterilization may be used.

Upon formulation, solutions will be administered in a manner compatible with the dosage formulation and in such amount as is therapeutically effective. For parenteral administration in aqueous solution, the solution may be suitably buffered if necessary. These particular solutions are especially suitable for intravenous, intramuscular, subcutaneous, and intraperitoneal administration. Some variation in dosage will necessarily occur depending on the condition of the subject being treated. Solutions containing bacteria will be administered in a manner compatible with appropriate safety precautions, and in a manner that takes into account the bacterial count of the preparation. The person responsible for administration will determine the appropriate dose for the individual subject.

An effective amount of a therapeutic or prophylactic polynucleotide or agent is determined based on the intended goal, for example regression of a tumor. The quantity to be administered, both according to number of treatments and dose, depends on the subject to be treated, the state of the subject and the protection desired. Precise amounts of the therapeutic or prophylactic composition also depend on the judgment of the practitioner and are peculiar to each individual.

In certain embodiments, it may be desirable to provide a continuous supply of a therapeutic composition to a patient. For various approaches, delayed release formulations could be used that provide limited but constant amounts of the therapeutic agent over an extended period of time. Continuous perfusion of the region of interest, such as a tumor or other area of diseased tissue, may be preferred. The administration could be post-operative, such as following tumor resection. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Construction of the YopE-LacI Fusion

Materials and Methods

The lacI gene was amplified from E. coli W3110 chromosomal DNA as a 1.1 kb fragment using primers SP044F (5′-GTGGGTACCGTGAAACCAGTAACG-3′ (SEQ ID NO:18)) and SP044R (5′-TAGGATCCGCTCACTGCCCGCTTT-3′ (SEQ ID NO:19)). The fragment was digested with KpnI and BamH1 and cloned in-frame downstream of a fragement containing the first 100 codons of yopE and the native yopE promoter from Yersinia enterocolitica into the vector pHSG575, yielding pSS350. Variants of this plasmid were created by removing the EcoR1-NdeI fragment encompassing the promoter region upstream of lacI and replacing it with the promoter and first 15 codons of yopE (pSS352), or with a fragment spanning the promoter and first 49 codons of yopE (pSS357). To generate a fusion protein with a smaller LacI domain, the first 334 codons of lacI were amplified by PCR from pSS350 and ligated to pSS356 digested with Kpn and BamHI, producing pSS357.

DNA binding activity of the fusion proteins was tested by measuring the ability of the fusion to repress LacZ expression. The lacI−, lacZ+ E. coli strain CSCG856 was transformed with each of the plasmids, and the repression of lacZ was measured by comparing Miller units in cells grown in the presence of lactose or in glucose. Fold repression of expression for each plasmid was: pSS350 (1.2-fold), pSS352 (>500-fold), pSS356 (16-fold), and pSS357 (16-fold).

Secretion of YopE-LacI via the TTSS was assessed by transforming a wild type and a TTSS- strain of Y. enterocolitica with pSS356 or pSS357. The cells were grown under conditions designed to induce secretion and the amount of the fusion protein in the culture supernatant was determined by immunoblot of the supernatant proteins using anti-LacI antiserum. The 45-kDa fusion protein encoded by pSS356 and the 42 kDa protein encoded by pSS357 were found in greater quantities in the supernatants of the Y. enterocolitica strains that had an intact TTSS compared to the strain that was TTSS minus.

Reporter constructs were based on vectors containing green fluorescent protein. The gfp sequence from pEGFP1 (Clontech) was digested with SspI and a chloramphenicol resistance genes was inserted. The cytomegalovirus (CMV) promoter was isolated as a 0.9 kb BglII-HindIII fragment from CMV and inserted into the BglII-HindIII digested plasmid to produce pCMV-EGFPΔ. pSS361 contains the CMV-EGFP fragment of pCMV-EGFPΔ and lacO cloned into pBR322.

To test for DNA secretion through the TTSS, the fusion protein expressing strains were transformed with pSS361 and tested under inducing and non-inducing conditions for their ability to secrete the lacO-containing plasmid into the culture supernatant. Induced cultures were incubated with a rabbit anti-LacI antibody and a biotinylated anti-rabbit secondary antibody. Streptavidin-containing paramagnetic beads were added to remove the DNA/YopE-LacI/antibody complexes. The culture medium contained more plasmid DNA in the presence of a functional translocation apparatus. Little or no plasmid DNA was detected in medium derived from a strain lacking the TTSS.

DNA translocation into eukaryotic cells via the TTSS was assessed using cultured Henle cells. Yersinia strains carrying pSS356 (YopE-LacI fusion vector) and/or pSS361 (eukaryotic GFP reporter construct) were added to Henle cell monolayers in the presence of cytochalasin D to prevent uptake of the bacteria. Gentamicin was added after 1 hour to kill the bacteria, and the cells were washed to remove the bacteria and cytochalasin D after an additional 90 minutes. The Henle cells were harvested after 48 hours and analyzed by flow cytometry to measure transfer and expression of the EGFP reporter gene. Cells exposed to Yersinia carrying an intact TTSS and both pSS346 and pSS361 showed expression of GFP.

### Example 2

### Construction of Regulated Lysis Vectors

The Shigella uhpT gene is specifically expressed within the intracellular environment of mammalian cells. This effect is mimicked in vitro by growth in the presence of 0.4% glucose-6-phosphate, the major form of glucose inside the mammalian cell. Other carbon sources do not allow expression of this gene. When fused to an open reading frame, the uhpT promoter directs the expression of that gene within the intracellular environment. A fragment containing the uhpT promoter was fused to the gene encoding T7 polymerase to create pFZ27. This plasmid is integrated into the attB site in a T7 polymerase resistant strain of Shigella flexenri to produce strain SF2019 or into standard laboratory strains of E. coli. The lysis construct consists of a plasmid carrying the T7 promoter fused to rabbit defensin. When grown in the presence of 0.4% glucose-6-phosphate, the strains are killed; survival is less than 1×10⁻⁴.

A pFZ27 (PuhpT-T7 pol construct in integrating vector) was also constructed. T7 RNA polymerase gene was amplified from BL21(DE3) by colony PCR and cloned into pGEMT-Easy vector (Promega). The PCR was carried out using Expand High Fidelity PCR System (Roche), with primers FZ017 (5′-CTGGAAGAGGCACTAAATGAAC-3′ (SEQ ID NO:20)) and FZ018 (5′-CCCTCTATAGTGAGTCGTATTG-3′ (SEQ ID NO:21)). The recombinant plasmid carrying the T7 RNA polymerase gene was designated pFZ19.

The S. flexneri uhpT promoter region was amplified from strain SA100 genomic DNA as a 260 bp fragment with primers FZ007 (5′-GAAGATCTCGATACCTGGCACTGGA-3′ (SEQ ID NO:22)) and FZ008 (5′-TCGCCCGGGTTACTCCTGAAATGAA-3′ (SEQ ID NO:23)). This fragment was cloned into pGEMT-Easy vector to generate pFZ6, and pFZ6 was further verified by DNA sequencing. Then, the BglII/SpeI fragment from pFZ6, containing the uhpT promoter, was cloned into integrating vector pCD11PKS at the BglII/SpeI site. The resulting construct was designated pFZ20.

pFZ27 was constructed by cloning the PstI/SphI fragment from pFZ19, containing the T7 RNA polymerase gene, into pFZ20 at the PstI/SphI site. Upon induced with 0.4% G-6-P, the strain DH5αλpir (pFZ27+pFZ73) produced a higher level of β-galactosidase activity and the strain DH5αλpir (pFZ27+pFZ50) showed reduced CFU, indicating that pFZ27 contains inducible and functional T7 RNA polymerase gene (pFZ73 is a plasmid containing the E. coli lacZ gene controlled by the T7 promoter; pFZ50 is a plasmid containing synthetic rat defensin gene fused with pelB leader and ΦX174 lysis E gene, both under the control of the T7 promoter).

pFZ58 (modified PuhpT-T7 pol construct in integrating vector) was also constructed. To eliminate any possible interference of the T7 promoter located at the original cloning vector pCD11PKS, the 3.0 kb SpeI fragment from pFZ27, containing PuhpT-T7 pol, was polished with pfu (Stratagene) and cloned into pCD11PSK at the polished BssHII site, yielding pFZ58. Upon induced with 0.4% G-6-P, the strain DH5αλpir (pFZ58+pFZ73) produced a higher level of β-galactosidase activity and the strain DH5αλpir (pFZ58+pFZ50) showed reduced CFU, indicating that pFZ58 contains inducible and functional T7 RNA polymerase gene (pFZ73 is a plasmid containing the E. coli lacZ gene controlled by the T7 promoter; pFZ50 is a plasmid containing synthetic rat defensin gene fused with pelB leader and ΦX174 lysis E gene, both under the control of the T7 promoter).

Still further, pFZ72 ((PuhpT-T7 pol construct in replicating vector) was constructed. The 3.0 kb BglII/HindIII fragment from pFZ27, containing PuhpT-T7 pol, was cloned into pACYC184 at the BamHI/HindIII site, yielding pFZ72.

### Example 3

### Lysis Gene Constructs

A pFZ49 (synthetic rat defensin gene NP-1 fused with pelB leader under the control of the T7 promoter) was constructed. Primers FZ001 (5′-GGATCCGGTGACCTGCTACTGTCGTCGTACTCGTTGCGGTTTCCGTGAACGTC TGTCCGGTGCTTG-3′ (SEQ ID NO:24)) and FZ002 (5′-GTCGACTTAACGACAGCACAGACGGTAGATACGACCACGGTAACCGCAAGC ACCGGACAGACGTTC-3′ (SEQ ID NO:25)) were annealed and extended with Taq polymerase. The resulting product was digested with BamHI/SalI and cloned into pET25b at the BamHI/SalI site, yielding pFZ1. The XbaI/XhoI fragment from pFZ1, containing pelB-NP-1 fusion gene, was cloned into pET23a at the XbaI/XhoI site, yielding pFZ49.

A pFZ50 (synthetic rat defensin gene NP-1 fused with pelB leader and ΦX174 lysis E gene under the control of the T7 promoter) vector was also constructed. ΦX174 E gene, whose product is responsible for lysis, was PCR amplified from ΦX174 RF DNA with primers FZ013 (5′-AAGGCCTACTGACCGCTCTC-3′ (SEQ ID NO:26)) and FZ014 (5′-CGTGCATGCTTGCCTTTAGTACC-3′ (SEQ ID NO:27)) and cloned into pGEMT-Easy vector, yielding pFZ10. The NotI fragment from pFZ10, containing the ΦX174 E gene, was cloned into pFZ1 at the NotI site, yielding pFZ45. The XbaI/XhoI fragment from pFZ45, containing pelB-NP-1-E, was cloned into pET23a at the XbaI/XhoI site, yielding pFZ50. pFZ50 was verified by DNA sequencing, showing that the transcription orientation of E gene is the same as the pelB-NP-1 fusion gene, both under the control of the T7 promoter.

When pFZ49 or pFZ50 was introduced into an E. coli strain carrying an inducible T7 RNA polymerase gene, the recombinant strain displayed reduced CFU under the inducing condition.

### Example 4

### In Vivo Transduction

Mice will be infected orally, orogastrically, or intranasally with the bacteria carrying the delivery system and a reporter or therapeutic gene. The bacteria (10⁵ to 10⁹ bacteria) will be suspended in phosphate buffered saline and delivered in a volume of 5 to 100 μl or any volume therebetween. The expression of the reporter or therapeutic gene will be measured three or more days after delivery. An example of a reporter construct assay is gfp (encoding green fluorescent protein) fused to a eukaryotic promoter. Intestinal or pulmonary epithelium will be excised from the mouse three to five days after delivery of the bacteria. GFP production will be measured by fluorescence of transfected or transduced tissue. Some of the constructs will contain all or portions of mouse mammary tumor virus (MMTV) to target lymphocytes and mammary tissue. Monitoring of constructs containing MMTV will be by measuring deletion of Vb14 T cells. It is contemplated that over the course of three months following delivery of MMTV via the bacterial lysis delivery system, a significant reduction in the number of Vb14 positive T cells will be seen, as measured by sorting peripheral blood lymphocytes stained with a fluorescent labeled antibody against Vb14 using a fluorescence activated cell sorter.

The delivery of constructs containing portions of MMTV fused to a reporter gene, gfp, lacZ, or other genes producing a measurable gene product will also be tested. These will be delivered via the intestinal or respiratory tracts. Expression of the reporter gene will be measured in mammary tissue and other tissues in the mouse.

All of the fusion proteins, compositions, and methods disclosed and claimed herein can be made and executed without undue experimentation in light of the present disclosure. While the fusion proteins, compositions, and methods of this invention have been described in terms of preferred embodiments, it will be apparent to those of skill in the art that variations may be applied to the fusion proteins, compositions, and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 6,376,248
U.S. Provisional Patent 20020045587
Ausubel et al., In: Current Protocols in Molecular Biology, John, Wiley & Sons, Inc, New York, 1996.
Birtalan et al., Mol. Cell, 9(5):971-980, 2002.
Cheng et al., Mol. Microbiol., 24(4):757-765, 1997.
Console et al., Biol. Chem., 278(37):35109-35114, 2003.
Courvalin et al., C R Acad. Sci. III, 318(12):1207-1212, 1995.
Darji et al., FEMS Immunol. Med. Microbiol., 27(4):341-349, 2000.
Derossi et al., J. Biol. Chem., 269(14):10444-10450, 1994.
Dietrich et al., Nat. Biotechnol., 16(2):181-185, 1998.
Elliott and O'Hare, Cell, 88(2):223-233, 1997.
Galan and Bliska, Annu. Rev. Cell Dev. Biol., 12:221-255, 1996.
He et al., J. Biol. Chem., 273(33):20737-43, 1998.
Hense et al., Cell Microbiol., 3(9):599-609, 2001.
Kaniga et al., J. Bacteriol., 177(14):3965-3971, 1995b.
Kaniga et al., J. Bacteriol., 177(24):7078-7085, 1995a.
Kyte and Doolittle, J. Mol. Biol., 157(1):105-132, 1982.
Menard et al., J. Bacteriol., 175(18):5899-5906, 1993.
Menard et al., Trends Microbiol., 4(6):220-226, 1996.
Michiels and Cornelis, J. Bacteriol., 173(5):1677-1685, 1991.
Nagahara et al., Am. J. Physiol., 275(4 Pt 1):G740-G748, 1998.
Nakanishi et al., Curr. Protein Pept. Sci., 4(2):141-150, 2003.
Page and Parsot, Mol. Microbiol., 46(1):1-11, 2002.
Pelletier and Sonenberg, Nature, 334(6180):320-325, 1988.
Rosqvist et al., EMBO J., 13(4):964-972, 1994.
Salmond and Reeves, Trends Biochem. Sci., 18(1):7-12, 1993.
Sambrook et al., In: Molecular cloning, Cold Spring Harbor Laboratory Press, Cold Spring Harbor, N.Y., 2001.
Schesser et al., J. Bacteriol., 178(24):7227-7233, 1996.
Sigee and AL-Rabaee, Protoplasma, 130, 171-185, 1986.
Simonet and Falkow, Infect. Immun., 60(10):4414-4417, 1992.
Sizemore et al., Science, 270(5234):299-302, 1995.
Sory and Cornelis, Mol. Microbiol., 14(3):583-594, 1994.
Sory et al., Proc. Natl. Acad. Sci. USA, 92(26):11998-12002, 1995.
Stebbins and Galan, Nature, 414(6859):77-81, 2001.
Wagner et al., Science, 260:1510-1513, 1993.
Wattiau et al., Proc. Natl. Acad. Sci. USA, 91(22):10493-10497, 1994.
Wattiau and Cornelis, Mol. Microbiol., 8(1):123-131, 1993.
Weiss and Chakraborty, Curr. Opin. Biotechnol., 12(5):467-472, 2001.

What is claimed is:

1. A recombinant bacterium comprising:
(a) a first polynucleotide comprising an expression cassette comprising a first regulatable promoter that is operably linked to a polynucleotide encoding a recombinant RNA polymerase; and
(b) a second polynucleotide comprising an expression cassette having a second promoter driven by the recombinant RNA polymerase that is operably linked to a polynucleotide that when expressed lyse the bacterium,
wherein the bacterium is able to be internalized by a cell.

2. The bacterium of claim 1, further comprising a third polynucleotide that is a therapeutic polynucleotide or encodes a therapeutic polypeptide.

3. The bacterium of claim 2, wherein the polynucleotide encodes a tumor suppressor, an apoptosis inducer, an enzyme, an antibody, or a hormone.

4. The bacterium of claim 3, wherein the polynucleotide encodes Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

5. The bacterium of claim 2, wherein the polynucleotide is RNA.

6. The bacterium of claim 5, wherein the RNA is messenger RNA.

7. The bacterium of claim 5, wherein the RNA is antisense RNA.

8. The bacterium of claim 5, wherein the RNA is interfering RNA.

9. The bacterium of claim 5, wherein the RNA further comprises a ribozyme.

10. The bacterium of claim 2, wherein the polynucleotide is a DNA-RNA hybrid.

11. The bacterium of claim 1, wherein the bacterium is an attenuated non-pathogenic bacterium.

12. The bacterium of claim 11, wherein the attenuated bacterium is a Shigella species, a Yersinia species, a Salmonella species, or an E. coli species.

13. The bacterium of claim 1, wherein the first promoter is an inducible promoter.

14. A method of delivering at least one polynucleotide into a cell, comprising:
(a) obtaining a bacterium as described in claim 2; and
(b) contacting the cell with the bacterium.

15. The method of claim 14, wherein the first polynucleotide is a therapeutic polynucleotide or encodes a therapeutice polypeptide.

16. The method of claim 15, wherein the polynucleotide encodes a tumor suppressor, an apoptosis inducer, an enzyme, an antibody, or a hormone.

17. The method of claim 16, wherein the polynucleotide encodes Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

18. The method of claim 15, wherein the polynucleotide is RNA.

19. The method of claim 18, wherein the RNA is messenger RNA.

20. The method of claim 18, wherein the RNA is antisense RNA.

21. The method of claim 18, wherein the RNA is interfering RNA.

22. The method of claim 18, wherein the RNA further comprises a ribozyme.

23. The method of claim 15, wherein the polynucleotide is a DNA-RNA hybrid.

24. The method of claim 14, wherein the cell is in a human subject.

25. The method of claim 24, wherein the human subject is a patient with cancer.

26. The method of claim 24, wherein the human subject is a human subject at risk of developing cancer.

27. The method of claim 14, wherein the bacterium is an attenuated non-pathogenic bacterium.

28. The method of claim 27, wherein the attenuated bacterium is a Shigella species, a Yersinia species, a Salmonella species, or an E. coli species.

29. The method of claim 14, wherein the first promoter is active when the bacterium is internalized by the cell.

30. A fusion protein comprising:
(a) a Type III secretion domain; and
(b) a polynucleotide binding domain.

31. The fusion protein of claim 30, wherein the Type III secretion domain is a Shigella Type III secretion domain or a Yersinia Type III secretion domain.

32. The fusion protein of claim 31, wherein the Type III secretion domain is SEQ ID NO:3.

33. The fusion protein of claim 30, wherein the polynucleotide binding domain is a DNA binding domain.

34. The fusion protein of claim 30, wherein the polynucleotide binding domain is a sequence-specific polynucleotide binding domain.

35. The fusion protein of claim 34, wherein the sequence-specific binding domain is a Lac repressor DNA binding domain.

36. The fusion protein of claim 30, further comprising a polynucleotide bound to the polynucleotide binding domain.

37. The fusion protein of claim 36, wherein the polynucleotide is DNA.

38. The fusion protein of claim 37, wherein the polynucleotide is antisense DNA.

39. The fusion protein of claim 38, wherein the antisense DNA is antisense ras, antisense myc, antisense raf, antisense erb, antisense src, antisense fms, antisense jun, antisense trk, antisense ret, antisense gsp, antisense hst, antisense bcl, or antisense abl.

40. The fusion protein of claim 36, wherein the polynucleotide is RNA.

41. The fusion protein of claim 40, wherein the RNA is messenger RNA.

42. The fusion protein of claim 40, wherein the RNA is antisense RNA.

43. The fusion protein of claim 40, wherein the RNA is interfering RNA.

44. The fusion protein of claim 40, wherein the RNA further comprises a ribozyme.

45. The fusion protein of claim 36, wherein the polynucleotide is a DNA-RNA hybrid.

46. The fusion protein of claim 36, wherein the polynucleotide comprises one or more therapeutic or prophylactic polynucleotides.

47. The fusion protein of claim 46, wherein the therapeutic or prophylactic polynucleotide encodes a tumor suppressor, apoptotic agent, an enzyme, an antibody, or a hormone.

48. The fusion protein of claim 47, wherein the therapeutic or prophylactic polynucleotide encodes Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

49. A bacterium comprising the fusion protein of claim 36.

50. The bacterium of claim 49, wherein the bacterium is an attenuated non-pathogenic bacterium.

51. The bacterium of claim 49, wherein the bacterium is a Shigella species, or a Yersinia species.

52. A method of delivering at least one polynucleotide into a cell comprising:
(a) contacting the cell with a bacteria claim 49; and
(b) delivering the polynucleotide into the target cell via the TTSS of the bacterium.

53. The method of claim 52, wherein the cell is a mammalian cell.

54. The method of claim 53, wherein the mammalian cell is a human cell.

55. The method of claim 54, wherein the human cell is comprised in a human subject.

56. The method of claim 55, wherein the human subject is a patient with cancer.

57. The method of claim 55, wherein the human subject is a human subject at risk of developing cancer.

58. The method of claim 52, wherein the Type III secretion system secretion domain is a Shigella Type III secretion system domain, or a Yersinia Type III secretion system domain.

59. The method of claim 58, wherein the Type III secretion system domain is SEQ ID NO:3.

60. The method of claim 52, wherein the polynucleotide is DNA.

61. The method of claim 60, wherein the polynucleotide is antisense DNA.

62. The method of claim 61, wherein the antisense DNA is antisense ras, antisense myc, antisense raf, antisense erb, antisense src, antisense fms, antisense jun, antisense trk, antisense ret, antisense gsp, antisense hst, antisense bcl, or antisense abl.

63. The method of claim 52, wherein the polynucleotide is a DNA-RNA hybrid.

64. The method of claim 52, wherein the polynucleotide binding domain is a sequence-specific polynucleotide binding domain.

65. The method of claim 64, wherein the sequence-specific polynucleotide binding domain is a sequence-specific DNA binding domain.

66. The method of claim 65, wherein the sequence-specific DNA binding domain is a Lac repressor DNA binding domain.

67. The method of claim 52, wherein the polynucleotide encodes a therapeutic polypeptide.

68. The method of claim 67, wherein the polynucleotide encodes a tumor suppressor, an apoptosis inducer, an enzyme, an antibody, or a hormone.

69. The method of claim 68, wherein the polynucleotide encodes Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

70. The method of claim 52, wherein the polynucleotide is RNA.

71. The method of claim 70, wherein the RNA is messenger RNA.

72. The method of claim 70, wherein the RNA is antisense RNA.

73. The method of claim 70, wherein the RNA is interfering RNA.

74. The method of claim 70, wherein the RNA further comprises a ribozyme.

75. The method of claim 52, wherein the polynucleotide is a DNA-RNA hybrid.


SEQUENCE LISTING (27 sequences)

SEQ ID NO: 1 - DNA, 1055 bp, Yersinia pseudotuberculosis
gaattccccaactttgacaccgataaccggttcaatagtatctggaatagacaacgaaagttgttgaaataattgagtgatagcttgttcaaatgaatacattatgatctcataatagttagataaaatatcaacttaaccaaagcactctcggcagaccatcaattttagcctataatttttagtttttgttttgtctaatataacaacaaaaacagcagcgattttttatatagccatcggctattttcccactaagataaccttgttttaatagccaaggtaataaatagtcatgaaaatatcatcatttatttctacatcactgcccctgccgacatctgtgtcaggatctagcagcgtaggagaaatgtctgggcgctcagtctcacagcaaacaagtgatcaatatgcaaacaatctggccgggcgcactgaaagccctcagggttccagcttagccagccgtatcattgagaggttatcatcagtggcccactctgtgattgggtttatccaacgcatgttctcggaggggagccataaaccggtggtgacaccagcacccacacctgcacaaatgccaagtcctacgtctttcagtgacagtatcaagcaacttgctgctgagacgctgccaaaatacatgcagcagttgaatagcttggatgcagagatgctgcagaaaaatcatgctcagttcgctacgggcagcggccctcttcgtggcagtatcactcaatgccaagggctgatgcagttttgtggtggggaattgcaagctgaggccagtgccatcttaaacacgcctgtttgtggtattcccttctcgcagtggggaactattggtggggtggccagcgcgtacgtcgccagtggcgttgatctaacgcaggcagcaaatgagatcaaagggctggcgcaacagatgcagaaattactgtcattgatgtgatatggataaaaacaaggggatagtgtttccccctttttctatcaatattgcgaatatcttcgtccctgatctttcaggggcgaatcgttttttagcatgc

SEQ ID NO: 2 - protein, 219 aa, Artificial Sequence
  Description of Artificial Sequence  Synthetic Peptide
MKISSFISTSLPLPTSVSGSSSVGEMSGRSVSQQTSDQYANNLAGRTESPQGSSLASRIIERLSSVAHSVIGFIQRMFSEGSHKPVVTPAPTPAQMPSPTSFSDSIKQLAAETLPKYMQQLNSLDAEMLQKNHAQFATGSGPLRGSITQCQGLMQFCGGELQAEASAILNTPVCGIPFSQWGTIGGVASAYVASGVDLTQAANEIKGLAQQMQKLLSLM

SEQ ID NO: 3 - protein, 49 aa, Artificial Sequence
  Description of Artificial Sequence  Synthetic Peptide
MKISSFISTSLPLPTSVSGSSSVGEMSGRSVSQQTSDQYANNLAGRTES

SEQ ID NO: 4 - protein, 11 aa, Artificial Sequence
  Description of Artificial Sequence  Synthetic Peptide
YGRKKRRQRRR

SEQ ID NO: 5 - DNA, 306 bp, Artificial Sequence
  Description of Artificial Sequence  Synthetic Primer
atggatctggtagatcctagcctagagccctggaaccatccgggaagccagcctgcaactgcttgtaataagtgttactgtaaagtgtgttgctatcattgtcaagtttgctttctgaacaaaggcttaggcatctcctatggcaggaagaagcggagacaacgacgaagaactcctcaaagcagtaaggatcatcaaaatcctatacaaaagcaacccataccccgaacccaaggggtctcgacaggcccggaagaatcgaagaagaagatggagagcaaggcagagacagatcgattcgattag

SEQ ID NO: 6 - protein, 101 aa, Artificial Sequence
  Description of Artificial Sequence  Synthetic Peptide
MDLVDPSLEPWNHPGSQPATACNKCYCKVCCYHCQVCFLNKGLGISYGRKKRRQRRRTPQSSKDHQNPIQKQPIPRTQGVSTGPEESKKKMESKAETDRFD

SEQ ID NO: 7 - protein, 11 aa, Artificial Sequence
  Description of Artificial Sequence  Synthetic Peptide
YARAAARQARA

SEQ ID NO: 8 - protein, 16 aa, Artificial Sequence
  Description of Artificial Sequence  Synthetic Peptide
RQIKIWFQNRRMKWKK

SEQ ID NO: 9 - DNA, 1125 bp, Drosophila melanogaster
atgacgatgagtacaaacaactgcgagagcatgacctcgtacttcaccaactcgtacatgggggcggacatgcatcatgggcactacccgggcaacggggtcaccgacctggacgcccagcagatgcaccactacagccagaacgcgaatcaccagggcaacatgccctacccgcgctttccaccctacgaccgcatgccctactacaacggccaggggatggaccagcagcagcagcaccaggtctactcccgcccggacagcccctccagccaggtgggcggggtcatgccccaggcgcagaccaacggtcagttgggtgttccccagcagcaacagcagcagcagcaacagccctcgcagaaccagcagcaacagcaggcgcagcaggccccacagcaactgcagcagcagctgccgcaggtgacgcaacaggtgacacatccgcagcagcaacaacagcagcccgtcgtctacgccagctgcaagttgcaagcggccgttggtggactgggtatggttcccgagggcggatcgcctccgctggtggatcaaatgtccggtcaccacatgaacgcccagatgacgctgccccatcacatgggacatccgcaggcgcagttgggctatacggacgttggagttcccgacgtgacagaggtccatcagaaccatcacaacatgggcatgtaccagcagcagtcgggagttccgccggtgggtgccccacctcagggcatgatgcaccagggccagggtcctccacagatgcaccagggacatcctggccaacacacgcctccttcccaaaacccgaactcgcagtcctcggggatgccgtctccactgtatccctggatgcgaagtcagtttgaacgcaaacgcggaaggcagacatacacccggtaccagactctagagctagagaaggagtttcacttcaatcgctacttgacccgtcggcgaaggatcgagatcgcccacgccctgtgcctcacggagcgccagataaagatttggttccagaatcggcgcatgaagtggaagaaggagaacaagacgaagggcgagccgggatccggaggcgaaggcgacgagataacaccacccaacagtccgcagtag

SEQ ID NO: 10 - protein, 374 aa, Drosophila Antennapedia
MTMSTNNCESMTSYFTNSYMGADMHHGHYPGNGVTDLDAQQMHHYSQNANHQGNMPYPRFPPYDRMPYYNGQGMDQQQQHQVYSRPDSPSSQVGGVMPQAQTNGQLGVPQQQQQQQQQPSQNQQQQQAQQAPQQLQQQLPQVTQQVTHPQQQQQQPVVYASCKLQAAVGGLGMVPEGGSPPLVDQMSGHHMNAQMTLPHHMGHPQAQLGYTDVGVPDVTEVHQNHHNMGMYQQQSGVPPVGAPPQGMMHQGQGPPQMHQGHPGQHTPPSQNPNSQSSGMPSPLYPWMRSQFERKRGRQTYTRYQTLELEKEFHFNRYLTRRRRIEIAHALCLTERQIKIWFQNRRMKWKKENKTKGEPGSGGEGDEITPPNSPQ

SEQ ID NO: 11 - DNA, 750 bp, Herpes Simplex Virus
atgggggattctgaaaggcggaaatcggaacggcgtcgttcccttggatatccctctgcatatgatgacgtctcgattcctgctcgcagaccatcaacacgtactcagcgaaatttaaaccaggatgatttgtcaaaacatggaccatttaccgaccatccaacacaaaaacataaatcggcgaaagccgtatcggaagacgtttcgtctaccacccggggtggctttacaaacaaaccccgtgccaagcccggggtcagagctgtacaaagtaataaattcgctttcagtacggctccttcatcagcatctagcacttggagatcaaatacagtggcatttaatcagcgtatgttttgtggagcggttgcaactgtggctcaatatcacgcataccaaggcgcgctcgccctttggcgtcaagatcctccgcgaacaaatgaagaattagatgcatttctttccagagctgtcattaaaattaccattcaagagggtccaaatttgatgggggaagccgaaacctgtgcccgcaaactattggaagagtctggattatcccaggggaacgagaacgtaaagtccaaatctgaacgtacaaccaaatctgaacgtacaagacgcggcggtgaaattgaaatcaaatcgccagatccgggatctcatcgtacacataaccctcgcactcccgcaacttcgcgtcgccatcattcatccgcccgcggatatcgtagcagtgatagcgaataa

SEQ ID NO: 12 - protein, 249 aa, Artificial Sequence [sequence omitted]

SEQ ID NO: 13 - DNA, 1119 bp, Artificial Sequence
  Description of Artificial Sequence  Synthetic Primer
atgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggtttga

SEQ ID NO: 14 - protein, 359 aa, Artificial Sequence
  Description of Artificial Sequence  Synthetic Peptide
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESG

SEQ ID NO: 15 - DNA, 34 bp, Artificial Sequence
  Description of Artificial Sequence  Synthetic Primer
cacatgtggaattgtgagcggataacaatttgtg

SEQ ID NO: 16 - DNA, 1266 bp, Artificial Sequence
  Description of Artificial Sequence  Synthetic Primer
atgaaaatatcatcatttatttctacatcactgcccctgccgacatctgtgtcaggatctagcagcgtaggagaaatgtctgggcgctcagtctcacagcaaacaagtgatcaatatgcaaacaatctggccgggcgcactgaaagcatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggtttga

SEQ ID NO: 17 - protein, 408 aa, Artificial Sequence
  Description of Artificial Sequence  Synthetic Peptide
MKISSFISTSLPLPTSVSGSSSVGEMSGRSVSQQTSDQYANNLAGRTESMKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESG

SEQ ID NO: 18 - DNA, 24 bp, Artificial Sequence
  Description of Artificial Sequence  Synthetic Primer
gtgggtaccgtgaaaccagtaacg

SEQ ID NO: 19 - DNA, 24 bp, Artificial Sequence
  Description of Artificial Sequence  Synthetic Primer
taggatccgctcactgcccgcttt

SEQ ID NO: 20 - DNA, 22 bp, Artificial Sequence
  Description of Artificial Sequence  Synthetic Primer
ctggaagaggcactaaatgaac

SEQ ID NO: 21 - DNA, 22 bp, Artificial Sequence
  Description of Artificial Sequence  Synthetic Primer
ccctctatagtgagtcgtattg

SEQ ID NO: 22 - DNA, 25 bp, Artificial Sequence
  Description of Artificial Sequence  Synthetic Primer
gaagatctcgatacctggcactgga

SEQ ID NO: 23 - DNA, 25 bp, Artificial Sequence
  Description of Artificial Sequence  Synthetic Primer
tcgcccgggttactcctgaaatgaa

SEQ ID NO: 24 - DNA, 66 bp, Artificial Sequence
  Description of Artificial Sequence  Synthetic Primer
ggatccggtgacctgctactgtcgtcgtactcgttgcggtttccgtgaacgtctgtccggtgcttg

SEQ ID NO: 25 - DNA, 66 bp, Artificial Sequence
  Description of Artificial Sequence  Synthetic Primer
gtcgacttaacgacagcacagacggtagatacgaccacggtaaccgcaagcaccggacagacgttc

SEQ ID NO: 26 - DNA, 20 bp, Artificial Sequence
  Description of Artificial Sequence  Synthetic Primer
aaggcctactgaccgctctc

SEQ ID NO: 27 - DNA, 23 bp, Artificial Sequence
  Description of Artificial Sequence  Synthetic Primer
cgtgcatgcttgcctttagtacc